(19) United States
(12) Patent Application Publication
(10) Pub. No.: US 2007/0123520 A1
(43) Pub. Date: May 31, 2007

(54) METHYLENE BLUE THERAPY OF AVIAN INFLUENZA

(75) Inventors: Christopher Wood, Stoke Poges (GB); Robert C. Sterling, Nazareth, PA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Bioenvision, Inc.

(21) Appl. No.: 11/560,106

(22) Filed: Nov. 15, 2006

Related U.S. Application Data
(60) Provisional application No. 60/737,332, filed on Nov. 16, 2005.

(51) Int. Cl. A61K 31/5415

(52) U.S. Cl. 514/224.8

(57) ABSTRACT

A method for using thiazine dyes, especially methylene blue, alone or in combination with low levels of light, to treat or prevent avian influenza virus is described. Examples of useful thiazine dyes are methylene blue, azure A, azure C, toluidine, and thionine. The preferred dye is methylene blue, administered orally twice a day.

### CROSS-REFERENCE TO RELATED APPLICATION

This application claims priority under 35 U.S.C § 119 to U.S.S.No. 60/737,332 filed in the U.S, Patent and Trademark Office on Nov. 16, 2005, by Christopher Wood and Robert C. Sterling.

### FIELD OF THE INVENTION

This invention is generally in the area of methods for the treatment of viral diseases, and more specifically relates to the treatment of type A influenza viruses, specifically avian flu viruses, using thiazine dyes, and in particular methylene blue.

### BACKGROUND OF THE INVENTION

Avian flu is an infection caused by avian (bird) influenza (flu) viruses. These flu viruses occur naturally among birds. Wild birds worldwide carry the viruses in their intestines, but usually do not get sick from them. However, bird flu is very contagious among birds and can make some domesticated birds, including chickens, ducks, and turkeys, very sick and/or kill them. Avian influenza viruses may be transmitted from animals to humans in two main ways: directly by contact with birds or an avian virus-contaminated environment or through an intermediate host, such as a pig, which can be infected by both human and avian flu strains.

Avian flu viruses are type A influenza viruses and are divided into subtypes based on two proteins on the surface of the virus, hemagglutinin (HA) and neuraminidase (NA). There are 15 different HA subtypes and 9 different NA subtypes. Subtypes of influenza A virus are named according to their HA and NA surface proteins. For example, an “H7N2 virus” designates an influenza A subtype that has an HA 7 protein and an NA 2 protein. Similarly an “H5N1” virus has an HA 5 protein and an NA 1 protein.

“Human flu viruses” are those subtypes that occur widely in humans. There are only three known A subtypes of human flu viruses (H1N1, H1N2, and H3N2). All known subtypes of A viruses can be found in birds. However, “bird flu” viruses are influenza A subtypes chiefly found in birds, which usually do not infect humans, even though they can. Symptoms of human infection with avian viruses have ranged from typical flu-like symptoms (fever, cough, sore throat and muscle aches) to eye infections, pneumonia, severe respiratory diseases (such as acute respiratory distress), and other severe and life-threatening complications. The symptoms of bird flu may depend on which virus caused the infection.

H5 and H7 subtypes of avian influenza A viruses can be classified as either highly pathogenic avian influenza (HPAI) or low pathogenic avian influenza (LPAI). Influenza H9 virus has been identified only as a LPAI form. This distinction is made on the basis of genetic features of the virus. HPAI is usually associated with high mortality in poultry. It is not certain how the distinction between “low pathogenic” and “highly pathogenic” is related to the risk of disease in people. HPAI viruses can kill 90 to 100% of infected chickens, whereas LPAI viruses cause less severe or no illness if they infect chickens. Because LPAI viruses can evolve into HPAI viruses, outbreaks of H5 and H7 LPAI are closely monitored by animal health officials. Each of these three avian influenza A viruses (H5, H7, and H9) theoretically can be partnered with any one of nine neuraminidase surface proteins; thus, there are potentially nine different forms of each subtype (e.g., H5N1, H5N2, H5N3, H5N9). H5 infections have been documented in humans, sometimes causing severe illness and death. At least three confirmed cases of H9 infection of humans has occurred. H7 infection in humans is rare, but can occur among people who have direct contact with infected birds.

Infected birds shed flu virus in their saliva, nasal secretions, and feces. Susceptible birds become infected when they have contact with contaminated excretions or surfaces that are contaminated with excretions. It is believed that most cases of bird flu infection in humans have resulted from contact with infected poultry or contaminated surfaces. The risk from bird flu is generally low to most people because the viruses occur mainly among birds and do not usually infect humans. However, the current outbreak of avian influenza A (H5N1) among poultry in Asia and Europe is an example of a bird flu outbreak that has caused human infections and deaths.

In 1997, the first instance of direct bird-to-human spread of influenza A (H5N1) virus was documented during an outbreak of avian influenza among poultry in Hong Kong. The virus caused severe respiratory illness in 18 people, six of whom died. During late 2003 and early 2004, outbreaks of highly pathogenic avian influenza A (H5N1) occurred among poultry in 8 countries in Asia: Cambodia, China, Indonesia, Japan, Laos, South Korea, Thailand, and Vietnam. At that time, more than 100 million birds either died from the disease or were destroyed in an attempt to prevent further spread of the disease. From Dec. 30, 2003 to Mar. 17, 2004, 12 confirmed human cases of avian influenza A (H5N1) were reported in Thailand and 23 in Vietnam, resulting in 23 deaths. By late February 2004, the number of new human H5N1 cases being reported in Thailand and Vietnam slowed and then stopped.

Beginning in late June 2004, new outbreaks of lethal avian influenza A (H5N1) infection among poultry were reported by several countries in Asia: Cambodia, China, Indonesia, Malaysia, Thailand, and Vietnam. Since May 2005, outbreaks of H5N1 disease have been reported among poultry in Russia, China, Kazakhstan, Turkey, and Romania. Mongolia has reported outbreaks of H5N1 in wild, migratory birds. In October 2005, H5N1 was reported among migrating swans in Croatia.

During August to October 2004, sporadic human cases of avian influenza A (H5N1) were reported in Vietnam and Thailand. Since December 2004, a resurgence of poultry outbreaks and human cases has been reported in Vietnam. In February 2005, the first of four human cases of H5N1 infection from Cambodia was reported. In July 2005, the first human case of H5N1 in Indonesia was reported. Indonesia has continued to report human cases in August, September, October, and November 2005, thailand also reported new human cases of H5N1 in October 2005 and Vietnam reported a new human case in November 2005.

According to the Centers for Disease Control (CDC), the avian influenza A (H5N1) outbreak in Asia is not expected to diminish significantly in the short term. It is likely that H5N1 infection among birds has become endemic to the region and that human infections resulting from direct contact with infected poultry will continue to occur. It is believed that most cases of H5N1 infection in humans have resulted from contact with infected poultry, uncooked poultry products, or contaminated surfaces. So far, no sustained human-to-human transmission of the H5N1 virus has been identified, and no evidence for genetic reassortment between human and avian influenza A virus genes has been found; however, the outbreak in Asia continues to pose an important public health threat.

There is little preexisting natural immunity to H5N1 infection in the human population. If these H5N1 viruses gain the ability for efficient and sustained transmission among humans, an influenza pandemic could result, with high rates of illness and death. In addition to confirmed outbreaks of H5N1 virus, there have been confirmed instances of the avian influenza viruses H9N2, H7N2, H7N7 and H7N3 infecting humans.

Four different influenza antiviral drugs (amantadine, rimantadine, oseltamivir, and zanamivir) are approved by the U.S. Food and Drug Administration (FDA) for the treatment of influenza; three are approved for prophylaxis. All four have activity against influenza A viruses. However, the H5N1 virus currently infecting birds in Asia that has caused human illness and death is resistant to amantadine and rimantadine. Oseltamavir and zanamavir may be effective to treat the H5N1 virus, but additional studies still need to be done to prove their effectiveness.

It is therefore an object of the present invention to provide methods and compositions for treatment or prevention of avian influenza viral infections.

It is a further object of the present invention to provide methods and compositions for relatively inexpensive treatment of avian influenza viral infections.

### SUMMARY OF THE INVENTION

A method for using thiazine dyes, especially methylene blue, alone or in combination with low levels of light, to selectively inactivate or inhibit avian influenza viruses is described. Examples of useful thiazine dyes are methylene blue, azure A, azure C, toluidine, and thionine. The preferred dye at this time is methylene blue. Since methylene blue absorbs in the red wavelengths, i.e., approximately 670 nm, which penetrates tissue much better than other lower wavelengths, light penetrating the skin to the capillaries at the surface can be used to enhance the activity of the dye.

### DETAILED DESCRIPTION OF THE INVENTION

I. Therapeutic Compositions

A. Thiazine Dyes

Examples of useful thiazine dyes are methylene blue, azure A, azure B, azure C, methylene green, new methylene blue, Taylor's Blue, Toluidine Blue O, and thionine. Methylene blue is the preferred dye. These dyes are all commercially available from a number of different sources. Symmetrical 3,7-bis(dialkylamino)phenothiazin-5ium derivatives which may be useful are described in Moura et al., Current Drug Targets, Vol. 4, 133-141 (2003).

### Methylene Blue And Its Derivatives

Methylene blue, 3,7-Bis(dimethylamino)-phenothiazin-5-ium chloride, C₁₆H₁₈ClN₃S, is a dark green or blue thiazine dye which was first isolated in 1876. Methylene blue is a thiazine dye occurring as dark blue-green crystals which is soluble in water and sparingly soluble in alcohol, forming deep blue solutions. Methylene blue injectable has a pH of 3-4.5. The pKₐ is between 0 and −1.

Methylene blue has been approved for oral administration and has been reported to be effective as an antiseptic disinfectant, and antidote for cyanide and nitrate poisoning. Methylene blue, injected i.v. at a dose of 1 mg/kg body weight, is effective in the treatment of methemoglobinemia, a clinical disorder where more than 1% of the hemoglobin in the blood has been oxidized to Fe³⁺. Drug Facts and Comparisons, page 1655 (J. B. Lippincot Co., St. Louis, Mo. 1989) reports that methylene blue is useful as a mild genitourinary antiseptic for cystitis and urethritis, in the treatment of idiopathic and drug-induced methemoglobemia and as an antidote for cyanide poisoning. Recommended dosages are 55 to 130 mg three times daily, administered orally. Oral absorption is 53% to 97%, averaging 74%, DiSanto and Wagner, J. Pharm. Sci. 61(7) 1086-1090 (1972). Pharmacopeia states that the recommended dose is 50 to 300 mg by mouth; 1 to 4 mg/kg body weight i.v. Side effects include blue urine, occasional nausea, anemia and fever. American Hospital Formulary Service “Drug Information 88” states that the recommended i.v. dosage for children is 1 to 2 mg/kg body weight, injected slowly over several minutes, which can be repeated after an hour. 55 mg tablets are available from Kenneth Manne. 65 mg tablets are available from Star Pharmaceuticals. Methylene Blue Injection (10 mg/ml) is available from American Reagent, Harvey, Kissimmee, Pasadena.

Narsapur and Naylor reported in J. Affective Disorders 5, 155-161 (1983) that administration of methylene blue orally, at a dosage of 100 mg b.i.d. or t.i.d., or intravenously, 100 mg, infused over 10 min, may be effective in treating some types of mental disorders in humans, indicating that the dye may cross the blood-brain barrier and therefore have particular applicability in the treatment of viral infections of the brain and central nervous systems. Methylene blue was administered for periods of one week to 19 months to adult humans, with minimal side effects.

The American Hospital Formulary Service “Drug Information 88” reports that methylene blue is absorbed well from the GI tract, with about 75% excreted in urine and via the bile, mostly as stabilized colorless leukomethylene blue. As reported by G. E. Burrows in J. Vet. Pharmacol. Therap. 7, 225-231 (1984), the overall elimination rate constant of methylene blue, in sheep, is 0.0076±0.0016 min⁻¹, with minimal methemoglobin production at doses as high as 50 mg/kg and no hematologic changes seen up to four weeks after a total dose of 30 mg/kg methylgene blue. The 24 h LD₅₀ for intravenous methylene blue administered as a 3% solution was 42.3 mg/kg with 95% confidence interval limits of 37.3 to 47.9 mg/kg, demonstrating that methylene blue can be safely administered at a dosage of up to at least 15 mg/kg. As reported by Ziv and Heavner in J. Vet. Pharmacol. Therap. 7, 55-59 (1984) methylene blue crosses the blood-milk barrier easily.

U.S. Pat. No. 6,346,529 to Floyd, et al., describes the use of methylene blue and other thiazine dyes to inactivate HIV. It also demonstrates that the effect of the dye on different types of viruses is unpredictable, and that one cannot use results with one virus to predict efficacy with another. See Table 4, comparing efficacy against HIV with a lack of efficacy against Herpes Simplex Virus type 1 and type 2.

In contrast, U.S. Pat. No. 5,545,516 to Wagner describes the inactivation of extracellular enveloped viruses in blood and blood components by phenthiazin-5-ium dyes plus light. The described process in activates pathogenic contaminants in whole blood, plasma, cellular blood components, by adding a phenthiazin-5-ium dye(s) thereto and irradiating the dye-containing composition with light of wavelengths from 560 to 800 nm or red light, such that they are suitable for transfusion. Obviously the conditions for treating blood products in a laboratory, and the availability of a radiant light source are quite different from the conditions required to treat a patient with a viral conditions such as avian flu infection.

The compounds described herein have the chemical formula shown below: 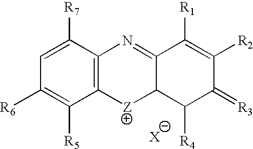
where R₁, R₂, R₄, R₅, and R₇ are independently selected from the group consisting of hydrogen, linear, branched or cyclic alkyl, aryl, substituted aryl, alkoxy, thioalkoxy, alkylamino, nitro, amino and halogen; R₃ and R₆ are independently selected from the group consisting of —O, —NH₂, —NHR₈, and —NR₉R₁₀ wherein R₈-R₁₀ is a linear, branched or cyclic hydrocarbon or R₉ and R₁₀ together with the nitrogen atom to which they are attached form an optionally substituted 5-, 6-, or 7-membered ring; wherein X is a counterion and wherein Z is either S or O.

Examples of useful thiazine dyes include, but are not limited to, methylene blue, methyl methylene blue, dimethyl methylene blue, azure A, azure B, azure C, methylene green, new methylene blue, Taylor's Blue, Toluidine Blue O, and thionine. These dyes are all commercially available from a number of different sources. Symmetrical 3,7-bis(dialkylamino)phenothiazin-5-ium derivatives which may be useful are described in Moura et al., Current Drug Targets, Vol. 4, 133-141 (2003). Derivatives of methylene blue in which the methyl groups of methylene blue have been replaced with ethyl, n-butyl, n-pentyl, and n-hexyl groups are described in Mellish et al., Photochemistry and Photobiology, Vol. 75, No. 4, pp. 392-397 (2002). Finally, phenoxazine dyes, in which the sulfur atom of the thiazine ring is replaced by an oxygen atom, may also be used. Examples of phenoxazine dyes include Nile Blue and its derivatives.

methylene blue, 3,7-Bis(dimethylamino)-phenothiazin-5-ium chloride, C₁₆H₁₈ClN₃S, is a dark green or blue thiazine dye which was first isolated in 1876. Methylene blue is a thiazine dye occurring as dark blue-green crystals which is soluble in water and sparingly soluble in alcohol, forming deep blue solutions. Methylene blue injectable has a pH of 3-4.5. The pKₐ is between 0 and −1.

Methylene blue and its derivatives typically exist as the chloride or bromide salts; however, other anions can be used to stabilize the positive charge on the molecule. Suitable anions include inorganic anions such as sulfate, phosphate, nitrate, and nitrite; and organic anions such as acetate, propionate, succinate, glycolate, stearate, lactate, malate, tartarate, citrate, ascorbate, pamoate, maleate, hydroxymaleate, phenylacetate, glutamate, benzoate, salicylate, sulfanilate, 2-acetoxybenzoate, fumarate, tolunesulfonate, naphthalenesulfonate, methanesulfonate, ethane disulfonate, oxalate, and isethionate salts.

B. Combinations with Other Active Compounds

The activity of the dye can be enhanced further by irradiation with light or by derivatization with compounds such as antisense mRNA. The thiazine dye can also be provided in combination with other known antibiotics, anti-inflammatories, antifungals, and antivirals.

methylene blue or a derivative of methylene blue can be administered adjunctively with other active compounds such as analgesics, antibiotics, antifungals, antivirals, anti-inflammatory drugs, antipyretics, antidepressants, antilepileptics, antihistamines, antimigraine drugs, antimuscarinics, anxioltyics, sedatives, hypnotics, antipsychotics, bronchodilators, anti asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics.

C. Additives, Excipients and Carriers

Formulations may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, and coating compositions.

Carrier also includes all components of the coating composition which may include plasticizers, pigments, colorants, stabilizing agents, and glidants. The delayed release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6^{th} Edition, Ansel et al., (Media, Pa.: Williams and Wilkins, 1995). These references provide information on carriers, materials, equipment and process for preparing tablets and capsules and delayed release dosage forms of tablets, capsules, and granules.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including, sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp.)

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethlene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-beta-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The compounds can be administered as tablets, hard or soft shell capsules, suspensions or solutions. Devices with different drug release mechanisms described herein can be combined in a final dosage form comprising single or multiple units. Examples of multiple units include multilayer tablets, capsules containing tablets, beads, granules, etc. A controlled release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release forms and their combinations are types of controlled release dosage forms. A delayed release dosage form is one that releases a drug (or drugs) at a time other than promptly after administration. An extended release dosage form is one that allows at least a twofold reduction in dosing frequency as compared to that drug presented as a conventional dosage form e.g. as a solution or prompt drug-releasing, conventional solid dosage form).

Extended release formulations are generally prepared as diffusion or osmotic systems, for example, as described in “Remington—The science and practice of pharmacy” (20the ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

Delayed release formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxpropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit®. (Rohm Pharma; Westerstadt, Germany), including Eudragit®. L30D-55 and L100-55 (soluble at pH 5.5 and above), Eudragit®. L-100 (soluble at pH 6.0 and above), Eudragit®. S (soluble at pH 7.0 and above, as a result of a higher degree of esterification), and Eudragits®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stablizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

The thiazine dyes can also be delivered using techniques known to those skilled in the art of drug delivery to target specific cell types or to enhance the activity of the dye. For example, a procedure utilizing injection of photoactive drugs four cancer treatment is described by Edelson, et al., in New England J. Med. 316, 297-303 (1987). Thiazine dyes can be specifically delivered to macrophages, a site of high type A virus concentration in patients, using techniques such as liposome delivery. Liposomes are generally described by Gregoriadis, Drug Carriers in Biology and Medicine Ch 14, 287-341 (Academic Press, NY, 1979). Methods for making light sensitive liposomes are described by Pidgeon, et al., in Photochem. Photobiol, 37, 491-494 (1983). Liposome compositions are commercially available from companies such as the Liposome Company, Inc., Princeton, N.J. Release of compounds from liposomes ingested by macrophages is described by Storm, et al., in Biochim. Biophys. Acta 965, 136-145 (1988).

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating, a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert), or the like. For detailed information concerning materials, equipment and processes for preparing tablets and delayed release dosage foes, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6. sup. th Ed. (Media, Pa.: Williams & Wilkins, 1995).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation also be prepared by any one of a number of conventional techniques, typically starting from a fluid dispersion. For example, a typical method for preparing drug-containing beads involves dispersing or dissolving, the active agent in a coating suspension or solution containing pharmaceutical excipients such, as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads

Alternatively, the dye can be continuously delivered to a patient over an extended period of time using a controlled release polymeric implant. Polymeric implants are generally manufactured from polymers which degrade in vivo over a known period of time. Examples of useful polymers include polyanhydrides, polylactic acid, polyorthoester, and ethylene vinyl acetate. These devices are also commercially available. Alza Corporation, Palo Alta, Calif., and Nova Pharmaceuticals, Baltimore, Md., both manufacture and distribute biodegradable controlled release polymeric devices.

D. Combination Therapies

The thiazine dye can be provided in combination with other known antibiotics, anti-inflammatories, antifungals, and antivirals to provide a combination therapy. Combination therapy is intended to include any chemically compatible combination of thiazine dye with other compounds, as long as the combination does not eliminate the activity of the thiazine dye.

For example, the thiazine dye can be used in combination with one or more other therapeutic agents, such as anti-neoplastic, anti-viral, anti-fungal, amoebicidal, trichomonocidal, analgesic, anti-neoplastic, anti-hypertensives, anti-microbial and/or steroid drugs, to treat antiviral infections. Suitable antibiotics include, but are not limited to, beta-lactam antibiotics, chloramphenicol, rifampin, clarithromycin, adriamycin, erythropoietin, neomycin, gramicidin, bacitracin, sulfonamides, and nalidixic acid. Suitable anti-inflammatory agents include, but are not limited to, cortisone, hydrocortisone, betamethasone, dexamethasone, fluocortolone, prednisolone, triamcinolone, indomethacin, sulindac. Suitable anti-fungals include, but are not limited to, voriconazole (Vfend®), azoles, imidazoles, polyenes, posaconazole, fluconazole, itraconazole, amphotericin B, 5-fluorocytosine, miconazole, and ketoconazole. Suitable antivirals include, but are not limited to, acyclovir, amantadine, rimantadine, nevirapine, cidofovir (Vistide. TM.), trisodium phosphonoformate (Foscarnet. TM.), famcyclovir, pencyclovir, valacyclovir, zidovuditne (AZT, Retrovir. TM.), didanosine (dideoxyinosine, ddl, Videx. TM.), stavudine (d4T, Zerit. TM.), zalcitabine (dideoxycytosine, ddC, Hivid. TM.), nevirapine (Viramune. TM.), lamivudine (Epivir. TM., 3TC), saquinavir (Invirase. TM., Fortovase. TM.), ritonavir (Norvir. TM.), nelfinavir (Viracept. TM.), efavirenz (Sustiva. TM.), abacavir (Ziagen. TM.), amprenavir (Agenerase. TM.) indinavir (Crixivan. TM.), ganciclovir, AzDU, delavirdine (Rescriptor. TM.), interferon, cyclovir, alpha-interferon, ribavirin, and interferon or combinations of ribavirin and interferon or beta globulin.

II. Methods of Treatment

A. Patients

Patients to be treated include any animal infected with, or at risk of being infected with, a type A influenza virus. In one embodiment the patient is a bird, more preferably a domesticated bird. In a preferred embodiment, the patient is a human.

Patients to be treated include patients who are non-responsive to treatment with traditional anti-viral medications, meaning they have a virus that is resistant to traditional anti-viral therapy. Drug resistance is the result of microbes, such as viruses, changing in ways that reduce or eliminate the effectiveness of drugs, chemicals, or other agents to cure or prevent infections. Drug resistance can be classified into two categories, intrinsic or acquired. Drug resistance is considered intrinsic when viruses are intrinsically not sensitive to the drug (i.e. the parasite was never sensitive to the drug). Drug resistance is considered acquired when a normally sensitive virus acquires resistance to the drug (i.e. the virus is no longer sensitive to what is normally considered a toxic dose of the drug). Methylene blue and its analogues as described herein can be used to treat patients with drug resistant viruses, especially avian flu viruses, in instances of intractability to normal therapy.

B. Dosages

The drug, is preferably administered orally, although it can also be administered by injection. The preferred dosage range for methylene blue is 30 to 180 mg twice a day, more preferably between 60 and 130 mg twice a day, or a dosage which yields blood levels between 0.2 and 2000 and more preferably between 2 and 200 μM methylene blue, administered orally in an immediate release formulation. The appropriate in vivo dosage can be determined by extrapolation from in vitro levels, assuming the usual blood volume for adult humans is approximately 10, and taking into account the 74% oral absorption and 75% excretion of that absorbed over a period of time, and assuming the lower therapeutic index in darkness than in light.

The drug can be administered as long as is necessary to clear the viral infection, which can be from days to months to years. Suitable lengths of treatment for viral infections include, but are not limited to, one week, two weeks, three weeks, six weeks, 12 weeks (about 3 months), or even longer as necessary. In a preferred embodiment, patients are treated from 3 days to 6 weeks. Longer treatment times are recommended for patients who have responded poorly to other anti-viral treatments, relapse patients, patients, with more than one viral infection, and patients with complications due to viral infections.

The method described herein does not require administration of exogenous light, although the results may be enhanced by exposure to light in addition to that normally transmitted through the skin. Exposure to light can occur with exposure to sun light, a tanning light, or even incandescent light.

Combination therapy can be sequential, meaning treatment with one agent first followed by treatment with a second agent, or it can be simultaneous, meaning treatment with both agents at the same time. If the combination therapy is sequential, administration of a second agent occurs within a reasonable time after administration of the first agent. If the combination therapy is simultaneous, both agents can be administered at the same time in the same dose or in separate doses. The exact regimen will depend on the severity of the disorder and the response to the treatment.

All publications cited are incorporated by reference.

Modifications and variations of the method to selectively, and in a controlled manner, inhibit specific viruses such as avian influenza viruses, and use thereof in the treatment of viral infections will be obvious to those skilled in the art from the foregoing detailed description. Such modifications and variations are intended to come within the scope of the appended claims.

We claim:

1. A method for treating avian influenza virus in a patient comprising
administering to the patient a formulation comprising an effective amount of a thiazine dye in a pharmaceutically acceptable carrier to prevent or alleviate the symptoms of influenza A virus.

2. The method of claim 1, wherein the influenza A virus is an avian influenza A virus.

3. The method of claim 1 further comprising enhancing the anti-viral activity of the dye by exposure to non-ionizing radiation.

4. The method of claim 1 wherein the thiazine dye is selected from the group consisting of methylene blue, toluidine blue O, azure A, azure B, azure C, and combinations and derivatives thereof.

5. The method of claim 4 wherein the dye is methylene blue.

6. The method of claim 1 wherein the dye is in combination with a pharmaceutically acceptable carrier for administration orally and is administered orally.

7. The method of claim 1 wherein the dye is in combination with a pharmaceutically acceptable carrier for injection and is administered by injection.

8. The method of claim 1 further comprising delivering the dye in a controlled release formulation.

9. The method of claim 1 wherein the dosage for an adult human is between 30 and 180 mg of thiazine dye orally per day.

10. The method of claim 1 further comprising providing the thiazine dye in combination with a compound selected from the group consisting of antibiotics, anti-inflammatories, antifungals, and antivirals.

11. The method of claim 1 wherein the individual is infected with avian influenza A virus.

12. The method of claim 1 wherein the individual is exposed to avian influenza A virus.

13. A composition for inhibiting avian influenza virus comprising an effective amount of a thiazine dye in a pharmaceutically acceptable carrier to prevent or alleviate the symptoms of influenza A virus when administered to a human or animal in need thereof.

14. The composition of claim 13 wherein the thiazine dye is selected from the group consisting of methylene blue, toluidine blue O, azure A, azure B, azure C, and combinations and derivatives thereof.

15. The composition of claim 14 wherein the thiazine dye is methylene blue.

16. The composition of claim 13 further comprising a pharmaceutically acceptable carrier for oral administration.

17. The composition of claim 13 in a formulation for administration to birds.

18. The composition of claim 13 in the form of a controlled release formulation.

19. The composition of claim 16 wherein the amount of the thiazine dye is suitable for delivery of a dosage of between 30 and 180 mg of thiazine dye per day.

20. The composition of claim 13 further comprising a compound selected from the group consisting of antibiotics anti-inflammatories, antifungals, and antivirals.
